(19) United States
(12) Patent Application Publication
(10) Pub. No.: US 2009/0085884 A1
(43) Pub. Date: Apr. 2, 2009

(54) CONTROLLER AND MEDICAL TREATMENT APPARATUS

(75) Inventor: Yasushi Hibi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/914,421

(22) PCT Filed: Feb. 22, 2006
(86) PCT No.: PCT/JP2006/303177

§ 371 Date: Nov. 14, 2007

(30) Foreign Application Priority Data
May 19, 2005 (JP) 2005-147045

(51) Int. Cl. G06F 3/041; A61B 8/14

(52) U.S. Cl. 345/173; 600/443

(57) ABSTRACT

A controller according to the present invention has a control portion. The control portion reads out a panel image that is previously stored in a panel image memory, displays the panel image on a liquid crystal monitor of a touch panel, inputs panel coordinates information that is from the touch panel, converts the panel coordinates information into a corresponding key code based on a key code conversion table stored in a conversion table memory, and outputs the result to a key code output portion. It is therefore possible to simplify a communication driver of an apparatus main unit to reduce a load in communication processing.

### TECHNICAL FIELD

The present invention relates to a controller having a touch panel function that controls medical equipment and a medical treatment apparatus.

### BACKGROUND ART

An ultrasonic diagnostic apparatus sends ultrasonic waves to a target region for diagnosis of a patient (test subject) and receives echo signals from the target region for diagnosis. When using the ultrasonic diagnostic apparatus, an ultrasonic probe is inserted into a treatment instrument insertion channel of an endoscope to deliver a distal end portion thereof to the region of interest and observes the degree of invasion of a tumor.

An ultrasonic diagnostic apparatus is already in practical use that obtains an in-vivo tomogram by irradiating an ultrasonic pulse in vivo and receiving a reflection wave from a biogenic tissue.

Further, in recent years an ultrasonic diagnostic apparatus has also been put into practical use that is provided with a Doppler function that, other than an in-vivo tomogram from a test subject, observes haemodynamics inside the test subject by utilizing a Doppler effect in which the frequency of a reflection wave deviates due to the velocity of a moving reflector.

A controller for setting the measurement conditions and the like is connected to these kinds of ultrasonic diagnostic apparatuses. For example, as shown in FIG. 15, a controller 100 comprises a touch panel 101 that includes a liquid crystal monitor portion having a touch sensor function as a pointing device, a hard key portion 102 including a plurality of mechanical switches, and a liquid crystal display switching key 103 that switches the screen of the touch panel 101.

Although the key setting for each mechanical switch of the hard key portion 102 is previously allocated, it is possible to perform flexible key setting at the touch panel 101 by changing the screen display, and the operation keys on the screen are operated using the touch sensor function.

As shown in FIG. 16, the controller 100 is connected for use to a host PC 110 comprising the main unit of the ultrasonic diagnostic apparatus.

The hard key portion 102 of the controller 100 is connected to a key code output portion 111. When a mechanical switch of the hard key portion 102 is pressed, a key code corresponding to the mechanical switch is outputted to the host PC 110 by the key code output portion 111. Upon input of such a key code, the host PC 110 executes processing corresponding to the key code.

Meanwhile, on the basis of a display image setting signal from the host PC 110, a display control portion 113 reads out a panel image that is previously stored in a panel image memory 112 and displays the panel image on a liquid crystal monitor of the touch panel 101. When an operation key on the panel image is pressed, panel coordinates information that represents the two-dimensional coordinates of the touch panel 101 is outputted to the host PC 110 from the touch panel 101 by a touch sensor function. Upon input of the panel coordinates information, the host PC 110 executes corresponding processing based on the panel coordinates information and the image information of the panel image.

The controller 100 is also provided with a control state monitor portion 114 that monitors the control state of the host PC 110. The control state monitor portion 114 controls the display control portion 113 to cause it to display a panel image of a state corresponding to the control state of the host PC 110 on the touch panel 101. When the liquid crystal display switching key 103 is pressed, the control state monitor portion 114 switches to a panel image of the state that corresponds to the control state of the host PC 110.

For example, the control state monitor portion 114 causes a panel image in accordance with the kind of ultrasonic probe connected to the main unit of the ultrasonic diagnostic apparatus, i.e. the host PC 110, or the connection state or the like to be displayed on the touch panel 101.

More specifically, as shown in FIG. 17, upon detecting that a key is pressed at step S100, the controller 100 determines at step S101 whether or not the detected key is a key on the touch panel 101.

When the detected key is a key on the touch panel 101, at step S102 the touch panel 101 extracts the two-dimensional coordinates of the pressed key, and at step S103 outputs panel coordinates information to the host PC 110.

When the detected key is not the one on the touch panel 101, at step S104 the controller 100 determines whether the detected key is the liquid crystal display switching key 103.

When the detected key is not the liquid crystal display switching key 103, it means that the detected key is a mechanical switch of the hard key portion 102. Therefore, at step S105, the key code output portion 111 confirms the key code corresponding to the mechanical switch of the hard key portion 102, and outputs the key code to the host PC 110 at step S106.

In the case of the liquid crystal display switching key 103, the control state monitor portion 114 confirms the control state of the host PC 110 at step S107, controls the display control portion 113 to execute display control in accordance with the control state of the host PC 110 at step S108, and outputs display information to the host PC 110 at step S109.

In this connection, irrespective of whether the liquid crystal display switching key 103 is pressed, the control state monitor portion 114 continuously monitors the control state of the host PC 110, and controls the display control portion 113 in accordance with the control state of the host PC 110.

As shown in FIG. 18, upon receiving a signal from the controller 100 at step S110, the host PC 110 determines at step S111 whether or not the received signal is a key code, When the received signal is a key code, the host PC 110 confirms the received key code at step S112, refers to an operation command table based on the key code to recognize the corresponding operation command at step S117, and executes processing corresponding to the operation command at step S118.

When the received signal is not a key code, that is, when the received signal is panel coordinates information, the host PC 110 confirms the received panel coordinates information at step S13, and confirms the current display information of the touch panel 101 at step S114. Subsequently, the host PC 110 refers to a key code conversion table based on the panel coordinates information and the display information at step 5115, confirms the corresponding key code at step S116, and then proceeds to step S117.

As an example, a touch panel device of an ultrasonic diagnostic apparatus disclosed, for example, in Japanese Patent Laid-Open No. 4-135545 may be disclosed.

However, in the conventional controller, when a touch panel and a hard key portion are provided together therein, since coordinates information is sent to the host PC from the touch panel and key codes are sent to the host PC from the hard key portion. Therefore, in order to perform signal processing with the same command system it is necessary to execute processing that converts the information from the touch panel into key codes based on coordinates information and display information.

More specifically, there is a problem that at the host PC it is necessary to provide both a communication driver that performs signal processing of signals from the touch panel and a communication driver that performs signal processing of signals from the hard key portion. As a result, the communications processing is complicated.

The present invention was made in view of the above described circumstances, and an object of the present invention is to provide a medical treatment apparatus and a controller that can simplify a communication driver at the apparatus main unit to reduce the load in communication processing.

### DISCLOSURE OF THE INVENTION

### Means for Solving the Problem

A controller according to claim 1 of the present invention that controls medical equipment and has touch panel means that detects coordinates information on a display panel that displays a panel image having image keys, comprising:

conversion means that converts the coordinates information of the image keys into key codes of a predetermined code system.

A medical treatment apparatus according to claim 5 of the present invention comprises:

an observation apparatus that observes a living body; and

a controller that is connected to the observation apparatus and controls the observation apparatus;

wherein the controller has:

touch panel means that detects the coordinates information on a display panel that displays a panel image having the image keys; and

conversion means that converts the coordinates information of the image keys into key codes of a predetermined code system.

### BRIEF DESCRIPTION OF THE DRAWINGS

FIG. 1 is a configuration diagram showing the configuration of an ultrasonic diagnostic apparatus according to Embodiment 1 of the present invention;

FIG. 2 is a view showing an appearance of a controller shown in FIG. 1;

FIG. 3 is a block diagram showing the configuration of the controller shown in FIG. 2;

FIG. 4 is a first view showing a panel image that is displayed on the touch panel shown in FIG. 2;

FIG. 5 is a second view showing a panel image that is displayed on the touch panel shown in FIG. 2;

FIG. 6 is a third view showing a panel image that is displayed on the touch panel shown in FIG. 2;

FIG. 7 is a view that illustrates coordinates information of image keys of the panel image shown in FIG. 4;

FIG. 8 is a view that illustrates coordinates information of image keys of the panel image shown in FIG. 5;

FIG. 9 is a view that illustrates coordinates information of image keys of the panel image shown in FIG. 6;

FIG. 10 is a view showing a key code conversion table that is stored in a conversion table memory shown in FIG. 3;

FIG. 11 is a flowchart that illustrates the operation of a controller shown in FIG. 3;

FIG. 12 is a flowchart that illustrates the operation of an ultrasonic observation apparatus shown in FIG. 3;

FIG. 13 is a view showing the configuration of a modification example of the controller shown in FIG. 1;

FIG. 14 is a block diagram showing the configuration of the controller shown in FIG. 13;

FIG. 15 is a view showing the appearance of a conventional controller;

FIG. 16 is a block diagram showing the configuration of the controller shown in FIG. 15;

FIG. 17 is a flowchart that illustrates the operation of the controller shown in FIG. 17; and

FIG. 18 is a flowchart that illustrates the operation of a host PC shown in FIG. 17.

### BEST MODE FOR CARRYING OUT THE INVENTION

Hereunder, an embodiment of the present invention will be described with reference to the drawings.

### Embodiment 1

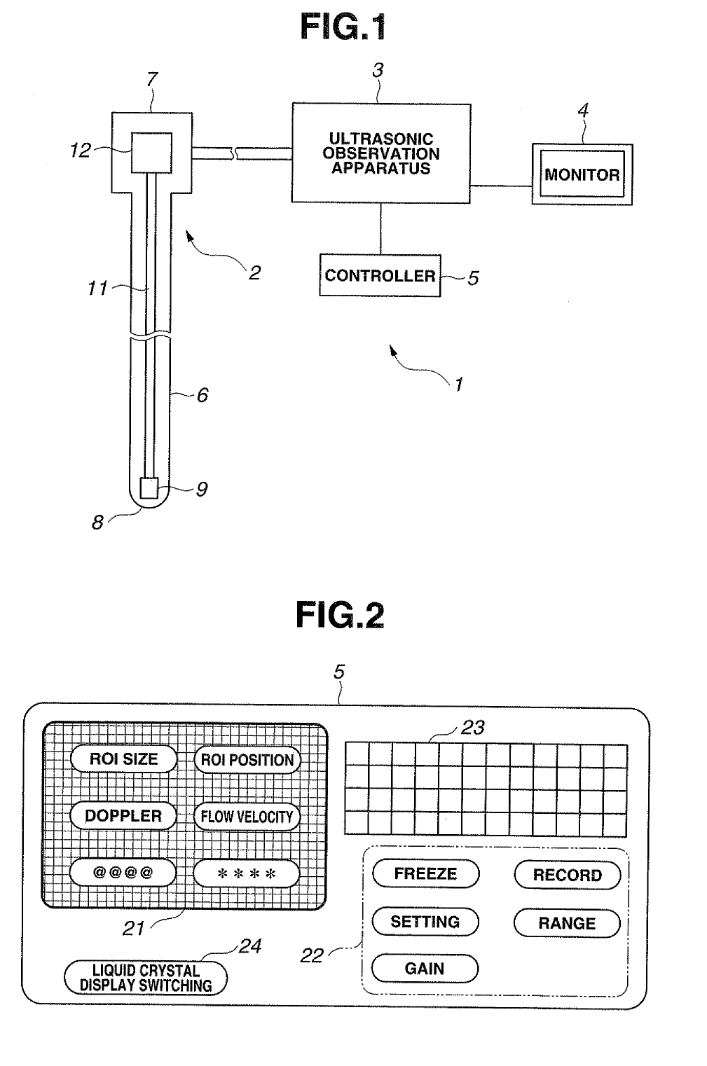

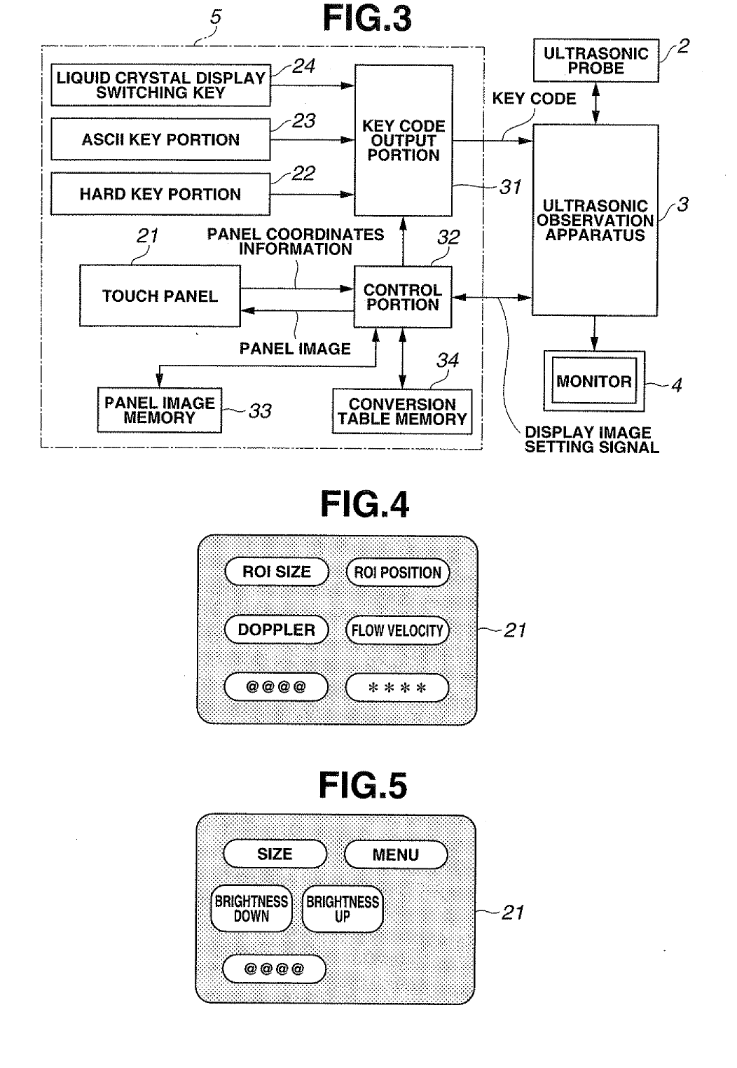

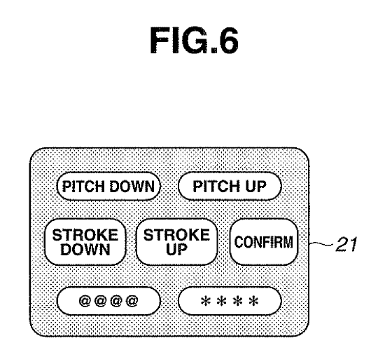

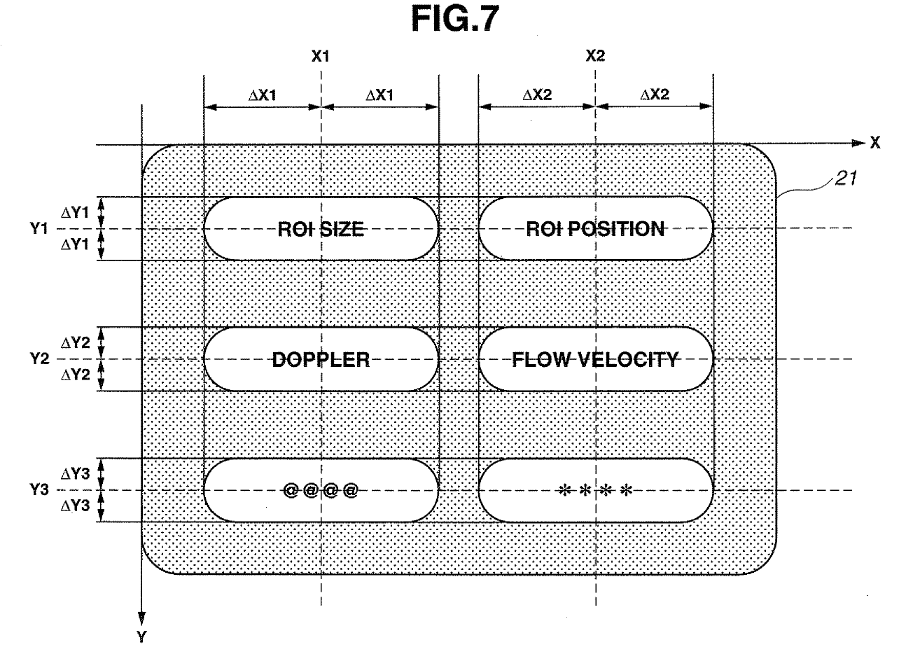

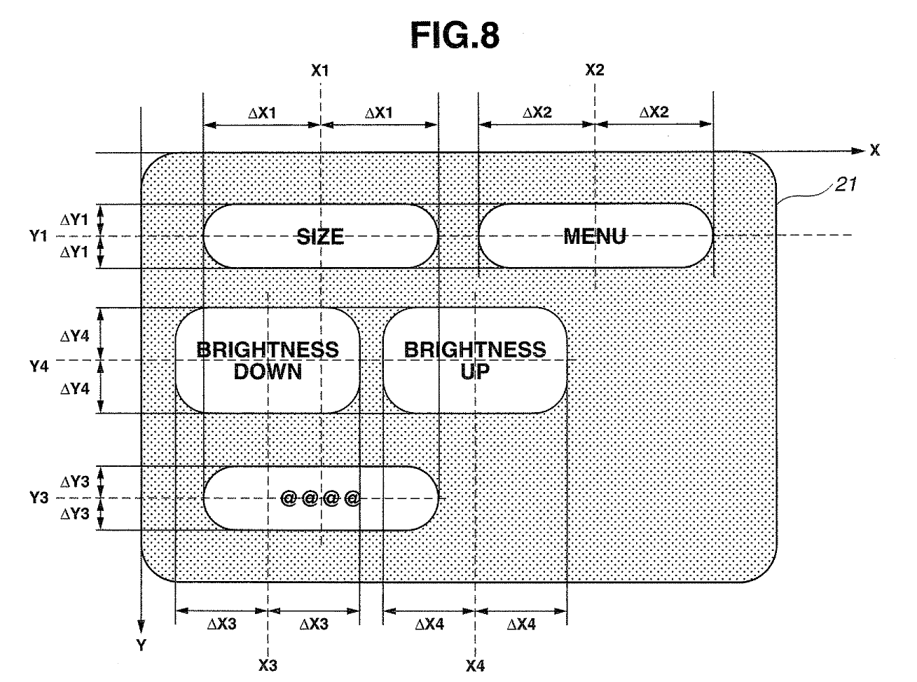

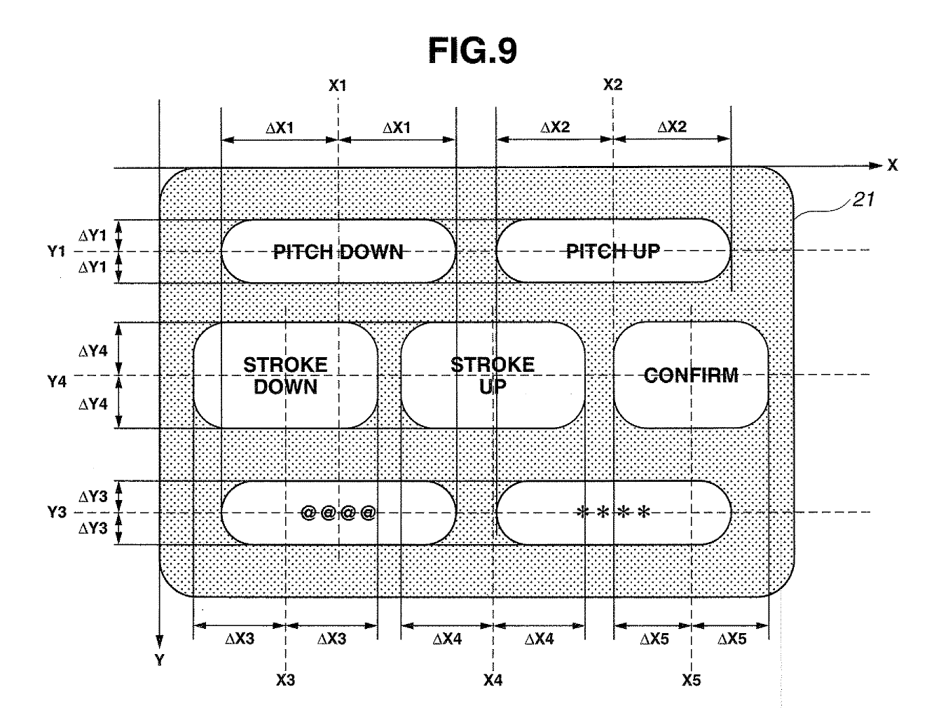

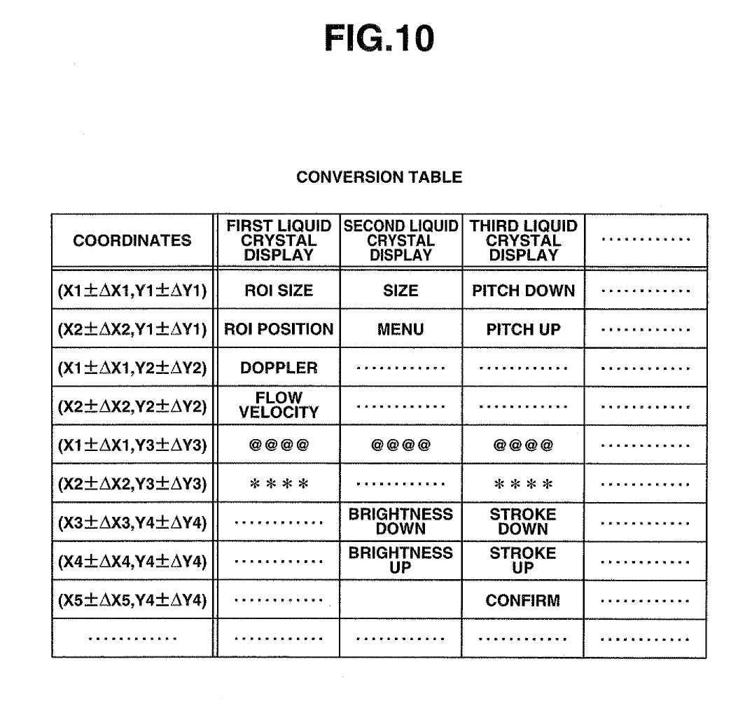

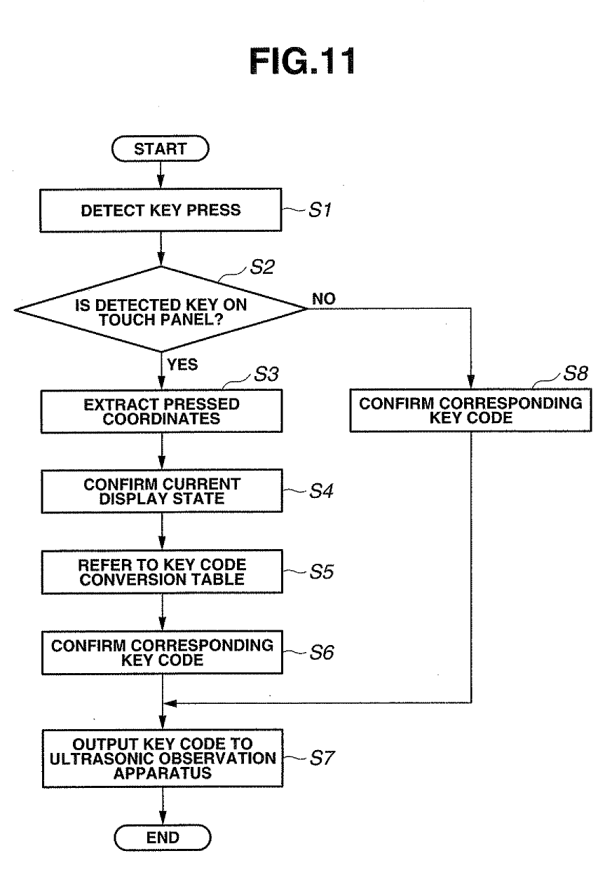

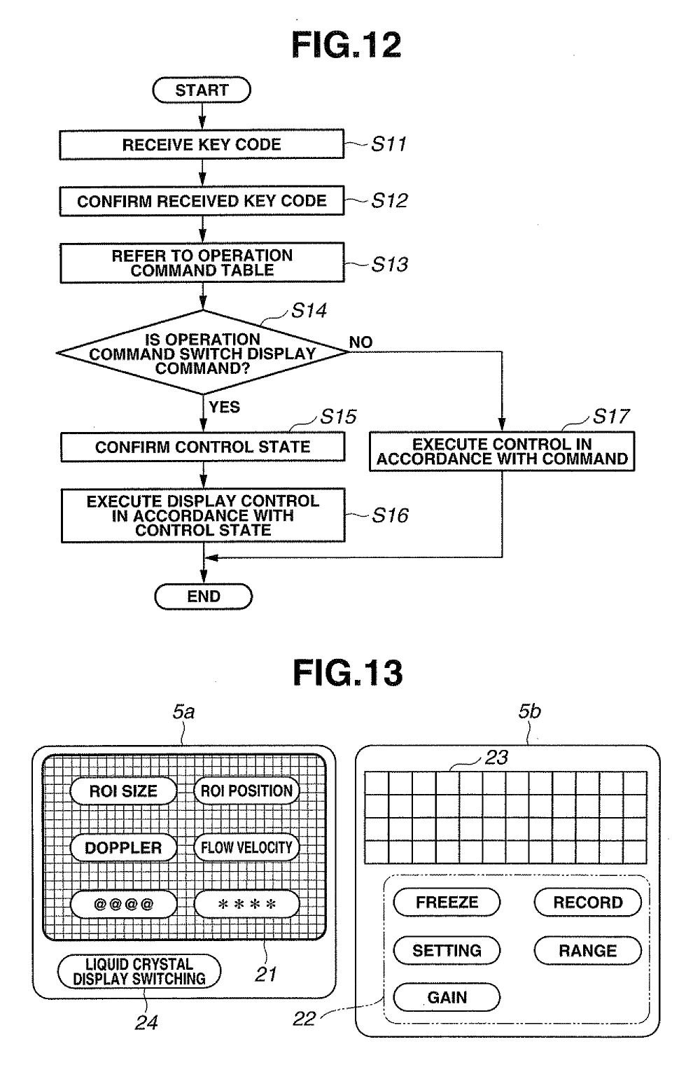

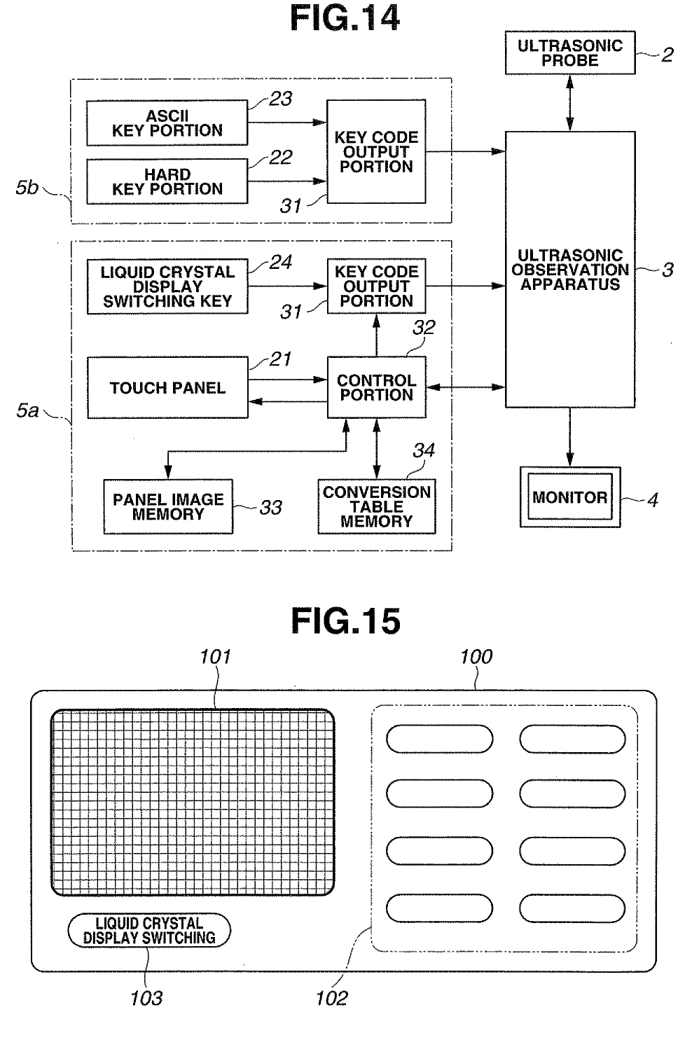

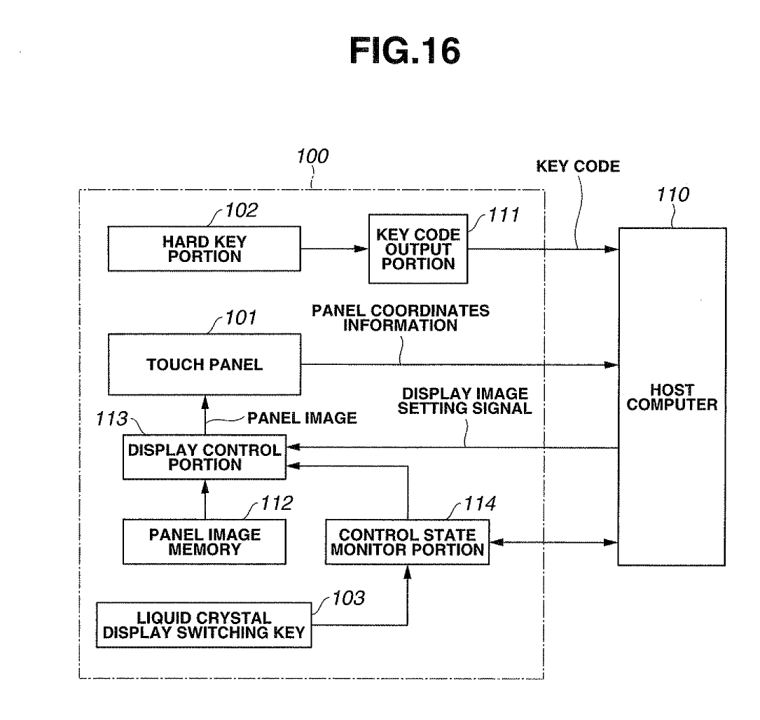

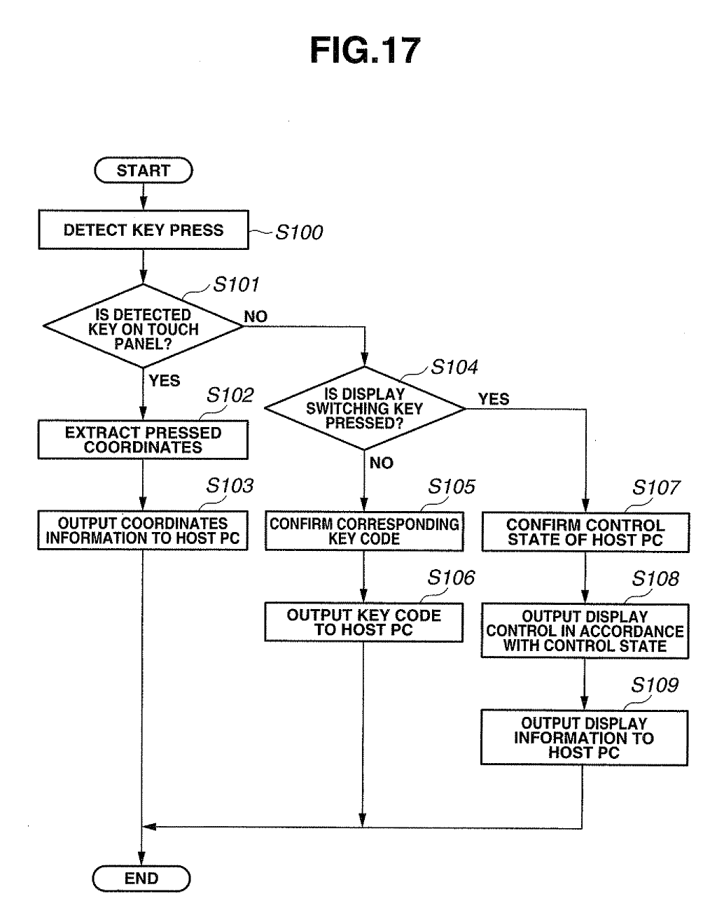

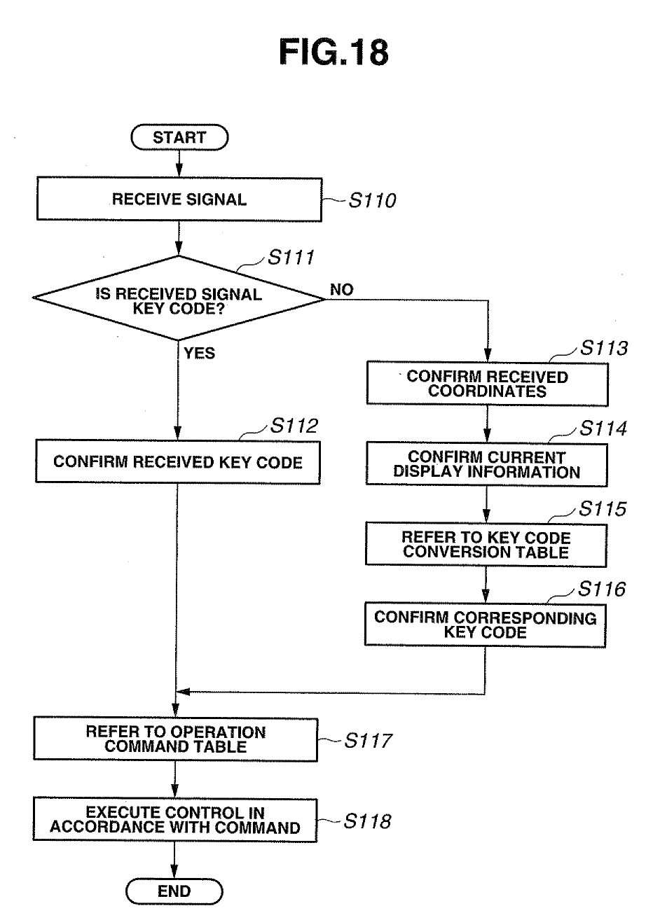

FIG. 1 to FIG. 14 relate to Embodiment 1 of the present invention. FIG. 1 is a configuration drawing showing the configuration of an ultrasonic diagnostic apparatus. FIG. 2 is a view showing the appearance of the controller shown in FIG. 1. FIG. 3 is a block diagram showing the configuration of the controller shown in FIG. 2. FIG. 4 is a first view showing a panel image that is displayed on the touch panel shown in FIG. 2. FIG. 5 is a second view showing a panel image that is displayed on the touch panel shown in FIG. 2. FIG. 6 is a third view showing a panel image that is displayed on the touch panel shown in FIG. 2. FIG. 7 is a view that illustrates coordinates information of image keys of the panel image shown in FIG. 4. FIG. 8 is a view that illustrates coordinates information of image keys of the panel image shown in FIG. 5. FIG. 9 is a view that illustrates coordinates information of image keys of the panel image shown in FIG. 6. FIG. 10 is a view showing a key code conversion table that is stored in a conversion table memory shown in FIG. 3. FIG. 11 is a flowchart that illustrates the operation of a controller shown in FIG. 3. FIG. 12 is a flowchart that illustrates the operation of an ultrasonic observation apparatus shown in FIG. 3. FIG. 13 is a view showing the configuration of a modification example of the controller shown in FIG. 1. FIG. 14 is a block diagram showing the configuration of the controller shown in FIG. 13.

As shown in FIG. 1, an ultrasonic diagnostic apparatus 1 comprises an ultrasonic probe 2 that is inserted inside the body of a test subject and sends and receives ultrasonic waves, an ultrasonic observation apparatus 3 to which the ultrasonic probe 2 is detachably connected and which performs processing to construct ultrasonic images, a monitor 4 that is a display device such as a CRT and a liquid crystal monitor that displays ultrasonic images by receiving video signals that are outputted from the ultrasonic observation apparatus 3, and a controller 5 that performs settings instructions and the like for the ultrasonic observation apparatus 3.

The ultrasonic probe 2 has an insertion portion 6 that is formed in a long and narrow shape so as to be easily inserted inside a test subject and an operation portion (grasping portion) 7 that is provided at the rear end of the insertion portion 6. An ultrasonic transducer array 9 formed of a plurality of transducer elements as ultrasonic transducers is built into a distal end portion 8 of the insertion portion 6.

The ultrasonic transducer array 9 is attached to a distal end of a flexible shaft 11 that passes through the inside of the insertion portion 6 of the ultrasonic probe 2. The proximal end of the flexible shaft 11 is connected to a rotary drive portion 12 that is provided in the operation portion 7.

As shown in FIG. 2, the controller 5 comprises a touch panel 21 comprising a liquid crystal monitor portion having a touch sensor function as a pointing device, a hard key portion 22 comprising a plurality of mechanical switches, an ASCII key portion 23 that inputs alphanumeric characters and the like, and a liquid crystal display switching key 24 that switches the screen of the touch panel 101.

As shown in FIG. 3, the liquid crystal display switching key 24, the hard key portion 22, and the ASCII key portion 23 of the controller 5 are connected to a key code output portion 31. When the liquid crystal display switching key 24 or a mechanical key of the hard key portion 22 or the ASCII key portion 23 is pressed, a key code corresponding to the mechanical key is outputted to the ultrasonic observation apparatus 3 by the key code output portion 31. Upon input of the key code to the ultrasonic observation apparatus 3, processing corresponding to the key code is executed.

The controller 5 also comprises a control portion 32 that reads out a panel image that is previously stored in the panel image memory 33 and displays the panel image on the liquid crystal monitor of the touch panel 21. The control portion 32 also inputs panel coordinates information from the touch panel 21, converts the panel coordinates information to a key code that corresponds to the panel coordinates information based on a key code conversion table, described later, that is stored in a conversion table memory 34, and outputs the resulting key code to the key code output portion 31. Upon input of such a key code, the ultrasonic observation apparatus 3 executes processing that corresponds to the key code.

Based on a display image setting signal from the ultrasonic observation apparatus 3, the control portion 32 controls panel images that are displayed on the liquid crystal monitor of the touch panel 21. For example, when the liquid crystal display switching key 24 is pressed and the corresponding key code is outputted to the ultrasonic observation apparatus 3 through the key code output portion 31, a display image setting signal is outputted to the control portion 32 from the ultrasonic observation apparatus 3. Subsequently, based on the display image setting signal, the control portion 32 reads out a plurality of kinds of panel images, as shown in FIG. 4 to FIG. 6, from predetermined addresses of the panel image memory 33, and outputs the panel images to the touch panel 21 to be displayed.

The display image setting signal from the ultrasonic observation apparatus 3 has status information that shows the control state of the ultrasonic observation apparatus 3. The control portion 32 carries out control of the display state of panel images based on the status information of the display image setting signal. More specifically, based on the status information, the control portion 32 displays panel images corresponding to the connection state or the type of the ultrasonic probe 2 that is connected to the ultrasonic diagnostic apparatus 3 and the like on the touch panel 21.

As shown in FIG. 7 to FIG. 9, the centre position of each image key on the plurality of kinds of panel images, for example the centre position (X1, Y1) at which the image key “ROI size” shown in FIG. 7 is displayed, is previously specified.

Therefore, when an image key region (X1±ΔX1, Y1±ΔY1) on the touch panel 21 with respect to a panel image that is a first liquid crystal display shown in FIG. 7 is pressed, the control portion 32 refers to the key code conversion table stored in the conversion table memory 34 shown in FIG. 10 to convert the coordinates information to a key code indicating that the “ROI size” image key is selected, and outputs the key code to the key code output portion 31.

The key code conversion for each image key of the panel image of a second liquid crystal display shown in FIG. 8 and the panel image of a third liquid crystal display shown in FIG. 9 is similarly performed by referring to the key code conversion table shown in FIG. 10.

In this case, the key codes corresponding to each mechanical key of the liquid crystal display switching key 24, the hard key portion 22, and the ASCII key portion 23 that are outputted from the key code output portion 31 and the key codes obtained upon the control portion 32 converting the coordinates information using the key code conversion table have the same code system, and the key code output portion 31 outputs these key codes with the same protocol to the ultrasonic observation apparatus 3.

Accordingly, at the ultrasonic observation apparatus 3, each mechanical key of the liquid crystal display switching key 24, the hard key portion 22, and the ASCII key portion 23 and each image key on the touch panel 21 can be recognized with the same communication driver.

Next, the processing of the controller 5 and the ultrasonic observation apparatus 3 that are configured in this manner will be described.

As shown in FIG. 11, at step S1, upon detecting that a mechanical key or an image key on the controller 5 is pressed, at step S2, the controller 5 determines whether or not the detected key is an image key on the touch panel 21.

When the detected key is an image key on the touch panel 21, the control portion 32 extracts the two-dimensional panel coordinates of the pressed key through the touch panel 21 at step S3, and checks the current display state (panel image) of the touch panel 21 at step S4. Subsequently, the control portion 32 refers to the key code conversion table stored in the conversion table memory 34 at step S5, confirms the key code corresponding to the two-dimensional panel coordinates and outputs the key code to the key code output portion 31 at step S6, and outputs the key code from the key code output portion 31 to the ultrasonic observation apparatus 3 at step S7.

When the detected key is not an image key on the touch panel 21, that is, when the detected key is a mechanical key on the controller 5, the key code output portion 31 generates a key code corresponding to the mechanical key at step S8, and that key code is outputted from the key code output portion 31 to the ultrasonic observation apparatus 3 at step S7.

As shown in FIG. 12, upon receipt of a key code at step S11 at the ultrasonic observation apparatus 3, the received key code is checked at step S12, and an unshown internal operation command table is referred to at step 53 to recognize the corresponding operation command.

Next, at step S14, it is determined whether or not the operation command is a command to switch the display from the liquid crystal display switching key 24. If the operation command is a command to switch the display, the control state of the ultrasonic observation apparatus 3 is confirmed at step S15, and at step S16 a display image setting signal is outputted to the controller to execute display control in accordance with the control state.

When the operation command is not a command to switch the display, that is, if the operation command is one corresponding to a mechanical key of the hard key portion 22 or the ASCII key portion 23, control in accordance with the operation command is executed at step S17.

In this connection, at the ultrasonic observation apparatus 3, the control portion 32 is controlled in accordance with the control state of the ultrasonic observation apparatus 3 regardless of whether or not the liquid crystal display switching key 103 is pressed.

According to the present embodiment as described above, with respect to, since the controller 5 converts key information of the image keys on the touch panel into key codes of a code system that is the same as the code system for key information of the mechanical keys, and outputs the key codes to the ultrasonic observation apparatus 3, signals from the controller 5 can be received with a single communication driver at the ultrasonic observation apparatus 3, thereby the communication processing load can be reduced.

Further, although conventionally the control state of the ultrasonic observation apparatus 3 is continuously monitored on the controller 5 side to control the display of the panel images of the touch panel 21, according to the present embodiment, since a display image setting signal that is in accordance with the control state is outputted directly from the ultrasonic observation apparatus 3 to the controller 5, there is also an advantage that means that monitors the control state of the ultrasonic observation apparatus 3 at the controller 5 is unnecessary.

Although according to the present embodiment a configuration is adopted in which the touch panel 21 and the hard key portion 22 and the like are provided in the controller 5, the present invention is not limited thereto. As shown in FIG. 13, a first controller 5a comprising the touch panel 21 and a second controller 5b comprising the hard key portion 22 and the like may be provided as separate members, and in this case also, as shown in FIG. 14, by applying the same code system for key codes that are outputted from the key code output portion 31 of the first controller 5a and key codes that are outputted from the key code output portion 31 of the second controller 5b, the same operations and advantages as the present embodiment can be obtained.

The present invention is not limited to the above described embodiment, and various changes and modifications and the like are possible without departing from the scope and spirit of the present invention.

1. A controller that has touch panel means that detects coordinates information on a display panel that displays a panel image having image keys, and that controls medical equipment, comprising:
conversion means that converts the coordinates information of the image keys into key codes of a predetermined code system.

2. The controller according to claim 1, comprising:
a mechanical key; and
key code generation means that, when the mechanical key is pressed, generates a key code of the code system that corresponds to the mechanical key.

3. The controller according to claim 2, wherein the mechanical key and the key code generation means are integrated with the touch panel means.

4. The controller according to claim 2, wherein the mechanical key and the key code generation means are separate members from the touch panel means.

5. A medical treatment apparatus, comprising:
an observation apparatus that observes a living body; and
a controller that is connected to the observation apparatus and controls the observation apparatus;
wherein the controller has:
touch panel means that detects coordinates information on a display panel that displays a panel image having image keys; and
conversion means that converts the coordinates information of the image keys into key codes of a predetermined code system.

6. The medical treatment apparatus according to claim 5, wherein the controller comprises:
a mechanical key; and
key code generation means that, when the mechanical key is pressed, generates a key code of the code system that corresponds to the mechanical key.

7. The medical treatment apparatus according to claim 6, wherein the mechanical key and the key code generation means are integrated with the touch panel means.

8. The medical treatment apparatus according to claim 6, wherein the mechanical key and the key code generation means are separate members from the touch panel means.

9. The medical treatment apparatus according to claim 5, wherein the observation apparatus is an ultrasonic observation apparatus.

10. The medical treatment apparatus according to claim 9, wherein the ultrasonic observation apparatus comprises:
an ultrasonic probe having an ultrasonic transducer at a distal end of an insertion portion that is inserted inside the living body;
an apparatus main unit that performs signal processing of ultrasonic signals that are obtained by the ultrasonic probe; and
display means that displays ultrasonic images that are subjected to signal processing at the apparatus main unit;
and wherein the controller is connected to the apparatus main unit.

11. The medical treatment apparatus according to claim 6, wherein the observation apparatus is an ultrasonic observation apparatus.

12. The medical treatment apparatus according to claim 7, wherein the observation apparatus is an ultrasonic observation apparatus.

13. The medical treatment apparatus according to claim 8, wherein the observation apparatus is an ultrasonic observation apparatus.

14. The medical treatment apparatus according to claim 11,
wherein the ultrasonic observation apparatus comprises;
an ultrasonic probe having an ultrasonic transducer at a distal end of an insertion portion that is inserted inside the living body;
an apparatus main unit that performs signal processing of ultrasonic signals that are obtained by the ultrasonic probe; and
display means that displays ultrasonic images that are subjected to signal processing at the apparatus main unit;
and wherein the controller is connected to the apparatus main unit.

15. The medical treatment apparatus according to claim 12,
wherein the ultrasonic observation apparatus comprises:
an ultrasonic probe having an ultrasonic transducer at a distal end of an insertion portion that is inserted inside the living body;
an apparatus main unit that performs signal processing of ultrasonic signals that are obtained by the ultrasonic probe; and
display means that displays ultrasonic images that are subjected to signal processing at the apparatus main unit;
and wherein the controller is connected to the apparatus main unit.

16. The medical treatment apparatus according to claim 13,
wherein the ultrasonic observation apparatus comprises:
an ultrasonic probe having an ultrasonic transducer at a distal end of an insertion portion that is inserted inside the living body;
an apparatus main unit that performs signal processing of ultrasonic signals that are obtained by the ultrasonic probe; and
display means that displays ultrasonic images that are subjected to signal processing at the apparatus main unit;
and wherein the controller is connected to the apparatus main unit.
